(19) United States
(12) Patent Application Publication
(10) Pub. No.: US 2014/0227311 A1
(43) Pub. Date: Aug. 14, 2014

(54) Method for Removing Immunosuppressive Properties of HIV Envelope Glycoproteins

(71) Applicants: Shervin Bahrami, Aarhus C (DK); Mogens Ryttergaard Duch, Risskov (DK)

(72) Inventors: Shervin Bahrami, Aarhus C (DK); Mogens Ryttergaard Duch, Risskov (DK)

(73) Assignee: SKAU APS, Aarhus N (DK)

(21) Appl. No.: 14/239,961

(22) PCT Filed: Aug. 15, 2012
(86) PCT No.: PCT/DK2012/050297

§ 371 Date: Feb. 20, 2014

(30) Foreign Application Priority Data
Aug. 23, 2011 (DK) PA 2011 70463

(51) Int. Cl. C07K 14/005; C07K 7/08

(52) U.S. Cl. CPC C07K 14/005; C07K 7/08; C12N 2770/00034
USPC 424/188.1; 530/395; 530/326; 530/324

(57) ABSTRACT

The present invention concerns a method for removal of immunosuppressive effects of envelope glycoproteins derived from human immunodeficiency virus, such as for vaccination purposes and for generation of neutralizing antibodies to HIV. The invention further concerns vaccines and antibodies obtainable by the method, as well as the use of such vaccines and antibodies.

The present invention relates to removal of immunosuppressive effects of envelope glycoproteins derived from human immunodeficiency virus for vaccination purposes and for generation of neutralizing antibodies to HIV.

### Technical Background

There are two major types of HIV; HIV-1 and HIV-2 that infect humans. SIV is a virus very similar to HIV which infects monkeys and has been found in African monkeys. They all belong to a group of retroviridae known as Lentiviruses. Retroviruses transfer their genes from a producer cell to a target cell as a genomic RNA transcript, which is reverse-transcribed after infection and integrated into the DNA genome of the target cell.

The first person with a documented HIV-infection died in 1959. In the early 1980s doctors in the US become aware that more and more patients suffered from abnormal infections and showed signs of immune failure. The syndrome was named Acquired Immune Deficiency Syndrome (AIDS) and it was soon after discovered that HIV was the causative agent for the observed destruction of the immune system.

Only attenuated (that is live but weakened) HIV-strains has been able to provide immunity in primate studies even though they will never reach a required safety profile suitable for mass vaccination. The replication process for HIV-1 has an error rate of about one per 10,000 base pairs. Since the entire viral genome is just under 10,000 base pairs, it is estimated that on average one error is introduced into the HIV-1 genome at each viral replication cycle. This high mutation rate contributes to extensive variability of the viruses inside anyone person and an even wider variability across populations. This variability has resulted in different HIV-1 variants being described, where these subspecies of virus are called “clades.” The distinctions are based on the structure of the envelope proteins, which are especially variable. The M (for major) variant is by far the most prevalent worldwide. Within the M variant are clades A, B, C, D, E, F, G H, I, J and K, with clades A through E representing the vast majority of infections globally. Clades A, C and D are dominant in Africa, while clade B is the most prevalent in Europe, North and South America and Southeast Asia. Clades E and C are dominant in Asia. These clades differ by as much as 35%. Another variant is Clade 0, which is observed in Cameroun isolates of HIV-1. The greatest variation in structure is seen in the envelope proteins gpl20 and gp41. There are two important results from the very high mutation rate of HIV-1 that have profound consequences for the epidemic. First, the high mutation rate is one of the mechanisms that allow the virus to escape from control by drug therapies. These new viruses represent resistant strains. The high mutation rate also allows the virus to escape the patient's immune system by altering the structures that are recognized by immune components. An added consequence of this extensive variability is that the virus can also escape from control by vaccines.

### SUMMARY OF THE INVENTION

Envelope glycoproteins derived from HIV with reduced immunosuppressive properties are antigens, which may have one or more of the following uses:

I) Providing a strong and persistent CD8⁺ T-cell response against HIV-virus glycoprotein in vivo
II) Generating neutralizing antibodies against HIV-virus in vivo
III) Providing an optimal antigen for vaccination against HIV
IV) Use as an antigen for generation of neutralizing antibodies against HIV

The strategy is based upon eliminating the inherent immunosuppressive properties of the envelope glycoprotein of HIV without disrupting the overall conformation of the envelope. This will be accomplished by rational introduction of specific amino acids changes into the HIV envelope glycoprotein.

The present invention relates to HIV-1 envelope polypeptides, to be used either as an antigen for HIV vaccination or as an antigen for generation of neutralizing antibodies against HIV. The invention also encompasses biological entities comprising such polypeptides or nucleic acids encoding those, in particular viral particles, viral like particles whether they are produced in vivo or in vitro, viral derived vector(s) or any eukaryotic expression vector(s). Moreover the invention relates to vaccine compositions, which comprise a polypeptide of the present invention and an adjuvant as well as a production method and kits comprising said vaccine compositions.

### General Disclosure

### Vaccination

Vaccination is the administration of antigenic material (a vaccine) to produce immunity to a disease.

### Mechanism of Function

Vaccinations involve the administration of one or more immunogens, which can be administered in several forms.

Some vaccines are administered after the patient already has contracted a disease. Vaccinia given after exposure to smallpox, within the first three days, is reported to attenuate the disease considerably, and vaccination up to a week after exposure likely offers some protection from disease or may modify the severity of disease. Other examples include experimental AIDS, cancer and Alzheimer's disease vaccines.

### Adjuvants and Preservatives

Vaccines typically contain one or more adjuvants, used to boost the immune response. Tetanus toxoid, for instance, is usually adsorbed onto alum. This presents the antigen in such a way as to produce a greater action than the simple aqueous tetanus toxoid.

Vaccines may further comprise preservatives, which are used to prevent contamination with bacteria or fungi. Preservatives may be used at various stages of production of vaccines.

### Types

All vaccinations work by presenting a foreign antigen to the immune system in order to evoke an immune response, but there are several ways to do this. The four main types that are currently in clinical use are as follows:
1. An inactivated vaccine consists of virus particles which are grown in culture and then killed using a method such as heat or formaldehyde. The virus particles are destroyed and cannot replicate, but the virus capsid proteins are intact enough to be recognized and remembered by the immune system and evoke a response. When manufactured correctly, the vaccine is not infectious, but improper inactivation can result in intact and infectious particles. Since the properly produced vaccine does not reproduce, booster shots are required periodically to reinforce the immune response.
2. In an attenuated vaccine, live virus particles with very low virulence are administered. They will reproduce, but very slowly. Since they do reproduce and continue to present antigen beyond the initial vaccination, boosters are required less often. These vaccines are produced by passaging virus in cell cultures, in animals, or at suboptimal temperatures, allowing selection of less virulent strains, or by mutagenesis or targeted deletions in genes required for virulence. There is a small risk of reversion to virulence, this risk is smaller in vaccines with deletions. Attenuated vaccines also cannot be used by immune-compromised individuals.
3. Virus-like particle vaccines consist of viral protein(s) derived from the structural proteins of a virus. These proteins can self-assemble into particles that resemble the virus from which they were derived but lack viral nucleic acid, meaning that they are not infectious. Because of their highly repetitive, multivalent structure, virus-like particles are typically more immunogenic than subunit vaccines (described below). The human papillomavirus and Hepatitis B virus vaccines are two virus-like particle-based vaccines currently in clinical use.
4. A subunit vaccine presents an antigen to the immune system without introducing viral particles, whole or otherwise. One method of production involves isolation of a specific protein from a virus or bacteria (such as a bacterial toxin) and administering this by itself. A weakness of this technique is that isolated proteins may have a different three-dimensional structure than the protein in its normal context, and will induce antibodies that may not recognize the infectious organism. In addition, subunit vaccines often elicit weaker antibody responses than the other classes of vaccines.
   A number of other vaccine strategies are under experimental investigation. These include DNA vaccination and recombinant viral vectors.

### Superiority of Virus Like Particles as Immunogens for Vaccination Purposes or for Generation of Neutralizing Anti Bodies

The development of a broad and neutralizing antibody response is vital for a protective HIV-1 vaccine. In addition, the induction of specific and effective cytotoxic T lymphocytes has been shown to be required for infection control. Thus, the developments of vaccine strategies that encompass both arms of the immune system are important. Differential MHC I and II antigen presentation is a key factor for initiation of a potent immune response. There appear to be short-comings in antigen presentation when antigens are administered solely as peptides or DNA. In contrast cross-talk between antigen presenting cells and T helper lymphocytes are promoted in vaccine strategies based on either infectious or non-infectious viral particles (virus like particles or VLPs). A potent induction of cell mediated immunity can be achieved even with whole-killed viral particles. This is likely a consequence of improved antigen uptake and systemic immunostimulation in macrophages and dendritic cells. Baculovirus-derived human immunodeficiency virus type i virus-like particle activate dendritic cels and induce ex vivo t-cell responses). To achieve potent immunogenic retroviral particles such as γ-retroviral particles or lentiviral like particles may be produced with HIV-1 envelope trimers on the surface. These particles can function as a superior immunogenic particle avoiding any risk associated with viral inactivation procedures. In a further development such VLPs can be produced by cells in the patient using appropriate vectors like but not restricted to vectors derived from vaccinia virus or measles virus

### Administration

A vaccine administration may be by any acceptable route, such as oral, by injection (intramuscular, intradermal, subcutaneous), by puncture, transdermal or intranasal.

### Vaccinia Virus (VACV or VV)

Poxviruses are unique among DNA virus because they replicate only in cytoplasm of the host cell, outside of the nucleus. Therefore, the large genome is required for encoding various enzymes and proteins involved in viral DNA replication and gene transcription. During its replication cycle, VV produces four infectious forms which differ in their outer membranes: intracellular mature virion (IMV), the intracellular enveloped virion (IEV), the cell-associated enveloped virion (CEV) and the extracellular enveloped virion (EEV). Although the issue remains contentious, the prevailing view is that the IMV consists of a single lipoprotein membrane, while the CEV and EEV are both surrounded by two membrane layers and the IEV has three envelopes. The IMV is the most abundant infectious form and is thought to be responsible for spread between hosts. On the other hand, the CEV is believed to play a role in cell-to-cell spread and the EEV is thought to be important for long range dissemination within the host organism.

### Common Strains

This is a list of some of the well-characterized vaccinia strains used in research and immunizations.
Western Reserve
Copenhagen
Dryvax (also known as “Wyeth”): the vaccine strain previously used in the United States, produced by Wyeth. It was replaced in 2008 by ACAM2000, produced by Acambis. It was produced as preparations of calf lymph which was freeze-dried and treated with antibiotics.
ACAM2000: The current strain in use in the USA, produced by Acambis. ACAM2000 was derived from a clone of a Dryvax virus by plaque purification. It is produced in cultures of Vero cells.
Modified vaccinia Ankara: a highly attenuated (not virulent) strain created by passaging vaccinia virus several hundred times in chicken embryo fibroblasts. Unlike some other vaccinia strains it does not make immunodeficient mice sick and therefore may be safer to use in humans who have weaker immune systems due to being very young, very old, having HIV/AIDS, etc.

Vaccinia viruses re-engineered to express foreign genes are robust vectors for production of recombinant proteins, the most common being a vaccine delivery system for antigens. Concerns about the safety of the vaccinia virus have been addressed by the development of vectors based on attenuated vaccinia viruses. One of them, the Modified Vaccinia Ankara (MVA) virus, is a highly attenuated strain of vaccinia virus that was developed towards the end of the campaign for the eradication of smallpox by Professor Anton Mayr in Germany. Produced by hundreds of passages of vaccinia virus in chicken cells, MVA has lost about 10% of the vaccinia genome and with it the ability to replicate efficiently in primate cells.

### Modified Vaccinia Ankara in Clinical Trials

MVA is widely considered as the vaccinia virus strain of choice for clinical investigation because of its high safety profile. MVA has been administered to numerous animal species including Monkeys, mice, swine, sheep, cattle, horses, and elephants, with no local or systemic adverse effects. Over 120,000 humans have been safely and successfully vaccinated against smallpox with MVA by intradermal, subcutaneous, or intramuscular injections.

### Challenge Studies in Primates

Immunization regimens incorporating priming with DNA vaccine and boosting with recombinant MVA-based vaccine have been found to provide some protection in non-human primates following challenge with an immunodeficiency virus. While vaccination did not prevent infection in these studies, it did result in lower viral load setpoints, increased CD4 counts, and reduced morbidity and mortality in vaccinated animals, compared to controls.

### Production of a Lentiviral VLPs by an MVA Vaccine in Vivo

A modified vaccinia virus Ankara (MVA) vaccine has been used in the SIV macaque therapeutic immunization model. MVA was selected as a vaccine vector, since it has previously shown to induce strong antiviral immune responses and protection. As viral antigens SIV Gag-Pol and Env were used.

### Neutralizing Antibody

A Neutralizing antibody, or NAb is an antibody which defends a cell from an antigen or infectious body by inhibiting or neutralizing any effect it has biologically. An example of a neutralizing antibody is diphtheria antitoxin, which can neutralize the biological effects of diphtheria toxin.

Most antibodies work by binding to an antigen, signaling to a white blood cell that this antigen has been targeted, after which the antigen is processed and consequently destroyed. The difference between neutralizing antibodies and binding antibodies is that neutralizing antibodies neutralize the biological effects of the antigen, while binding antibodies flag antigens.

This difference is what gives neutralizing antibodies the ability to fight viruses which attack the immune system, since they can neutralize function without a need for white blood cells (excluding production)

### Immunosuppressive Properties of Enveloped Viruses with Type I Fusion Proteins

Enveloped viruses enter their target cells by means of active fusion of their lipid membrane with that of the cells. The fusion is mediated by viral gene products in form of proteins embedded in the lipid membrane of the virus, the so-called viral fusion proteins [1].

At the same time fusion proteins of some enveloped viruses show an immune suppressive activity. Inactivated retroviruses are able to inhibit proliferation of immune cells upon stimulation [2-4]. Expression of these proteins is enough to enable allogenic cells to grow to a tumor in immune competent mice. In one study, introduction of ENV expressing construct into MCA205 murine tumor cells, which do not proliferate upon s.c. injection into an allogeneic host, or into CL8.1 murine tumor cells (which overexpress class I antigens and are rejected in a syngeneic host) resulted in tumor growth in both cases [5]. Such immunosuppressive domains have been found in a variety of different viruses with type 1 fusion mechanism such as Mason pfeizer monkey virus (MPMV), murine leukemia virus (MLV), lentiviruses such as HIV and in filoviruses such as Ebola and Marburg viruses [6-9].

The immune suppressive activity is located to a very well-defined structure within the class I fusion proteins. The immunosuppressive effects range from significant inhibition of lymphocyte proliferation [7,8], cytokine skewing (up regulating IL-10; down regulating TNF-α, IL-12, IFN-γ) [10] and inhibition of monocytic burst [11] to cytotoxic T cell killing [12]. Importantly, peptides spanning ISD are mostly active in these assays when either linked as dimers or coupled to a carrier (i.e. >monomeric). Such peptides derived from such immune suppressive domains are able to reduce or abolish immune responses such as cytokine secretion or proliferation of T-cells upon stimulation.

### Co-Location of the Immunosuppression Domain and the Fusion Domain

The immunosuppressive domain of retro-, lenti- and filoviruses overlap a structurally important part of the fusion subunits of the envelope proteins. Although the primary structure (sequence) of this part of the fusion proteins can vary greatly from virus to virus, the secondary structure, which is very well preserved among different virus families, is that of an alpha helix that bends in different ways during the fusion process This structure plays a crucial role during events that result in fusion of viral and cellular membranes. It is evident that the immunosuppressive domains of the class I fusion proteins overlap with a very important protein structure needed for the fusion proteins mechanistic function.

The energy needed for mediating the fusion of viral and cellular membranes is stored in the fusion proteins, which are thus found in a meta-stable conformation on the viral surface. Once the energy is released to drive the fusion event, the protein will find its most energetically stable conformation. In this regard fusion proteins can be compared with loaded springs that are ready to be sprung. This high energy conformation makes the viral fusion proteins very susceptible to modifications; Small changes in the primary structure of the protein often result in the protein to be folded in its stable post fusion conformation. The two conformations present very different tertiary structures of the same protein.

This presents a significant challenge if one wishes to remove the immunosuppressive effect without altering the tertiary structure of the fusion protein, but it has been shown in the case of simple retroviruses that small structural changes in the envelope protein are sufficient to remove the immune suppressive effect without changing structure and hence the antigenic profile, resulting in an antigen which is significantly more potent as a vaccine.

The non-immune suppressive envelopes are much better antigens for vaccination. The proteins can induce a 30-fold enhancement of anti-env antibody titers when used for vaccination [6]. Furthermore, viruses that contain the non-immunosuppressive form of the friend murine leukemia virus envelope protein, although fully infectious in irradiated immunocompromised mice cannot establish an infection in immunocompetent animals. Interestingly in the latter group the non-immunosuppressive viruses induce higher cellular and humoral immune responses, which fully protect the animals from subsequent challenge by wild type viruses [13]

### Further General Description of the Invention

The present invention provides a new approach to designing immunogenic HIV-1 envelope polypeptides derived from HIV-1 envelope protein, GP41 and/or GP120 and/or GP160 for vaccination purposes and/or for generation of neutralizing antibodies to HIV. Specific amino acid residues are mutated to repress the immunosuppression displayed by GP41 thereby boosting the immune response against HIV-1. Mutation of those specific residues however, must not completely destroy the overall protein structure of the viral envelope protein as e.g. measured functionally by fusion assays. The polypeptides of the present invention can be inserted in a suitable construct and expressed in an organism to produce a vaccine or an immunogenic response against HIV.

According to an aspect, the present invention concerns a method for making an HIV-1 envelope glycoprotein, which may be expressed on the surface of cells, virus or virus like particles, and which provides reduced immune suppression, allowing said HIV-1 envelope glycoprotein to generate an enhanced immune response as compared to wildtype; said method comprising the steps of:
Selecting at least one HIV envelope glycoprotein among the group consisting of GP41 and GP160;
Modifying the immunosuppressive domain of said envelope glycoprotein, said immunosuppressive domain being localizable by the sequence:

L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄
X₂₅X₂₆X₂₇X₂₈C₂₉

Where X is any amino acid residue;
Said modifying step comprising mutating or genetically modifying at least one of the amino acids selected among the amino acids X₂-X₁₇, whereby said sequence allows maintaining the ability of expression of said HIV-1 envelope glycoprotein on the surface of cells or virus, and further provides reduced immune suppression of said HIV-1 envelope glycoprotein, and wherein said amino acids after said modifying step are selected among:

X2: Q, R, N, A, G
X3: A, T, V, G, Q, R
X4: R, K, G, A, Q
X5: V, I, L, M, A, G, Q, R
X6: L, A, C, D, E, F, G, H, I, K, M, N, P, Q, R, S, T, V, W, Y
X7: A, T, S, G, Q, R
X8: V, I, L, Q, M, A, G, Q, R
X9: E, K, A, G, Q, R
X10: R, S, T, K, G, N, E, D, A, G, Q
X11: L, A, C, D, E, F, G, H, I, K, M, N, P, Q, R, S, T, V, W, Y
X12: L, V, I, M, R, A, G, Q
X13: K, Q, R, A, E, G, S, H, W, T, V
X14: D, E, N, H, G, A, G, Q, R
X15: Q, R, A, G
X16: Q, A, S, K, T, R, H, G
X17: L, M, S, Q, R, H, I, V, F, A, G

Said modifying step is not necessarily limited to modifying at least one of the amino acids selected among the amino acids X₂-X₁₇. As an example, even one, two, three or all of the amino acids L₁, G₂₂, C₂₃ and/or C₂₉ may be modified, as long as the obtained sequence allows maintaining the ability of expression of said HIV-1 envelope glycoprotein on the surface of cells or virus, and further provides reduced immune suppression of the obtained HIV-1 envelope glycoprotein. It is preferred that the amino acids L₁, G₂₂, C₂₃ and C₂₉ are not modified.

The term “localizable” is used to express that the relevant domain can be localized using the indicated sequence.

According to another aspect, the invention concerns an HIV-1 envelope glycoprotein obtainable according to the invention

According to an aspect, the invention concerns an antigen obtainable by selecting a part of an HIV-1 envelope glycoprotein, which HIV-1 envelope glycoprotein is obtainable according to the invention, said part comprising the modified domain of said envelope glycoprotein.

The expression “a part” in this context refers to any part.

According to an aspect, the invention concerns a nucleic acid sequence, preferably recombinant, encoding an HIV-1 envelope glycoprotein, an HIV-1 envelope polypeptide or an antigen according to the invention.

According to an aspect, the invention concerns an isolated eukaryotic expression vector comprising a nucleic acid sequence according to the invention

According to an aspect, the invention concerns a vaccine composition for HIV, comprising an HIV-1 envelope glycoprotein according to the invention.

According to an aspect, the invention concerns a method for producing an antibody, said method comprising the steps of:
Administering an entity selected among an HIV-1 envelope glycoprotein, an HIV-1 envelope polypeptide, an antigen, a nucleic acid sequence or a vector according to the invention to a host, such as an animal; and
Obtaining the antibody from said host.

According to an aspect, the invention concerns an antibody obtainable according to invention.

According to an aspect, the invention concerns neutralizing antibodies obtained or identified by the use of at least one HIV-1 envelope glycoprotein according to the invention.

According to an aspect, the invention concerns a method for manufacturing neutralizing antibodies comprising the use of at least one HIV-1 glycoprotein according to the invention.

According to an aspect, the invention concerns a method for manufacturing humanized neutralizing antibodies, comprising the use of at least one sequence selected among the sequences 1 to 336.

According to an aspect, the invention concerns a vaccine composition, comprising an entity selected among the group consisting of an HIV-1 envelope glycoprotein, an HIV-1 envelope polypeptide, an antigen, a nucleic acid sequence, a vector and an antibody according to the invention, and in addition at least one excipient, carrier or diluents.

According to an aspect, the invention concerns a method for producing a vaccine composition according to the invention comprising combining at least one HIV-1 envelope glycoprotein, an HIV-1 envelope polypeptide, an antigen, a nucleic acid sequence, a vector or an antibody according to the invention, preferably in purified form, with at least one adjuvant

According to an aspect, the invention concerns a pharmaceutical composition comprising an HIV-1 envelope glycoprotein, an HIV-1 envelope polypeptide, an antigen, a nucleic acid sequence, a vector, an antibody or a vaccine composition according to the invention, and at least one pharmaceutically acceptable excipient.

According to an aspect, the invention concerns a use of an HIV-1 envelope glycoprotein or an HIV-1 envelope polypeptide, or a dimerized version or a trimerized version or a multimerized version of the purified envelope polypeptide according to the invention as an antigen.

According to an aspect, the invention concerns a use of an HIV-1 envelope glycoprotein, an HIV-1 envelope polypeptide, an antigen, a nucleic acid sequence, a vector or an antibody according to the invention, for a medical purpose, such as for the treatment, amelioration or prevention of a clinical condition, such as for the manufacture of a medicament for the treatment, amelioration or prevention of a clinical condition, such as HIV.

According to an aspect, the invention concerns a method of treating or ameliorating the symptoms of an individual infected with HIV, or prophylactic treating an individual against HIV infection, comprising administering an amount of HIV-1 envelope glycoprotein, HIV-1 envelope polypeptide, antigen, nucleic acid sequence, vector or vaccine composition according to the invention.

In one aspect, the present invention relates to an HIV-1 envelope polypeptide which encompasses a region of 29 amino acids further comprising the mutated immunosuppressive domain rendering said immunosuppressive domain non-immunosuppressive where the amino acids at position 1, 22, 23 and 29 are invariable with the amino acid at position 1 being a Leucine, the amino acid at position 22 a Glycine, the amino acids at position 23 a Cysteine and the amino acid at position 29 a Cysteine. As such the HIV envelope antigen for making an HIV vaccine or generating neutralizing antibodies against HIV should contain the following sequence

<seqid 1;PRT;>:
L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄
X₂₅X₂₆X₂₇X₂₈C₂₉

Where X is any amino acid residue.

### DETAILED DISCLOSURE

In a preferred embodiment the antigen should encompass a sequence where the amino acids has been even more restricted to

<seqid 2;PRT;>:
L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉
where
X is
X1: L
X2: Q, R, N, A, G
X3: A, T, V, G, Q, R
X4: R, K, G, A, Q
X5: V, I, L, M, A, G, Q, R
X6: L, A, C, D, E, F, G, H, I, K, M, N, P, Q, R, S, T, V, W, Y
X7: A, T, S, G, Q, R
X8: V, I, L, Q, M, A, G, Q, R
X9: E, K, A, G, Q, R
X10: R, S, T, K, G, N, E, D, A, G, Q
X11: L, A, C, D, E, F, G, H, I, K, M, N, P, Q, R, S, T, V, W, Y
X12: L, V, I, M, R, A, G, Q
X13: K, Q, R, A, E, G, S, H, W, T, V
X14: D, E, N, H, G, A, G, Q, R
X15: Q, R, A, G
X16: Q, A, S, K, T, R, H, G
X17: L, M, S, Q, R, H, I, V, F, A
X18: L, M
X19: G, N, S, A, Q, R
X20: I, L, S, A, T, E, V, M, F
X21: W, F
X22: G
X23: C
X24: S, K, T, Q, R, A
X25: G, F, D, N, W
X26: K, N, R, F, M, Q, A, S
X27: L, H, P, A, Q, S, T, W, I, V, F, R
X28: I, V, T
X29: C

In yet a preferred embodiment the antigen should encompass a sequence where the amino acids has been even further restricted to

<seq id 3; PRT;>:
L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉
where
X is
X1: L
X2: Q, R, A, G, Q
X3: A, T, G, Q, R
X4: R, K, G, A, G, Q
X5: V, I, L, A, G, Q, R
X6: L, A, C, D, E, F, G, H, I, K, M, N, P, Q, R, S, T, V, W, Y
X7: A, S, T, G, Q, R
X8: V, I, L, M, A, G, Q, R
X9: E, K, A, G, Q, R
X10: R, S, T, K, G, A, Q
X11: L, A, C, D, E, F, G, H, I, K, M, N, P, Q, R, S, T, V, W, Y
X12: L, V, I, M, A, G, Q, R
X13: K, Q, R, A, E, G, S, H, W, T, V, G
X14: D, N, G, A, Q, R
X15: Q, R, A, G
X16: Q, K, T, R, H, A, G
X17: L, R, I, F, G, Q
X18: L, M
X19: G, N, S, R
X20: I, L, S, A, T, E, V, M, F
X21: W
X22: G
X23: C
X24: S, K, T, R, A
X25: G
X26: K, N, R
X27: L, H, P, A, Q, S, T, W, I, V, F, R
X28: I, V
X29: C

In an even more preferred embodiment the antigen should encompass a sequence where the amino acids has even been further restricted to

<seqid 4;PRT;>:
L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉
where
X is
X1: L
X2: Q, R, A, G
X3: A, T, G, Q, R
X4: R, K, A, G, Q
X5: V, I, L, A, G, Q, R
X6: L, A, C, D, E, F, G, H, I, K, M, N, P, Q, R, S, T, V, W, Y
X7: A, S, T, A, G, Q, R
X8: V, I, L, A, G, Q, R
X9: E, K, A, G, Q, R
X10: R, S, T, K, G, A, Q
X11: L, A, C, D, E, F, G, H, I, K, M, N, P, Q, R, S, T, V, W, Y
X12: L, V, I, M, A, G, Q, R
X13: K, Q, R, A, E, G, S, H, W, T, V, A, G
X14: D, N, A, G, Q, R
X15: Q, R, A, G
X16: Q, K, T, R, A, G
X17: L, R, I, F, A, G, Q
X18: L
X19: G, N, S, R
X20: I, L, S, V, F
X21: W
X22: G
X23: C
X24: S, K, T, A
X25: G
X26: K, R
X27: L, H, P, A, Q, S, T, W, I, V, F, R
X28: I, V
X29: C

Particularly preferred sequences are sequences selected among sequences with seqID 5 to seqID 228.

Examples of HIV envelopes suited as antigens for HIV vaccination or for generating neutralizing antibodies against HIV are given but not restricted to seqid 229-366. These sequences represent different HIV clades or combinations thereof, which an HIV vaccine must be effective against.

Another aspect relates to a method of treating, preventing or ameliorating a clinical condition, said method comprising administering to an individual suffering from said clinical condition an effective amount of an HIV-1 envelope polypeptide or part thereof as defined in the present invention, an antigen as defined in the present invention, a nucleic acid as defined in the present invention, a vector as defined in the present invention, a biological entity as defined in the present invention, a vaccine composition as defined in the present invention, or a kit-of-parts as defined in the present invention.

### Terms

HIV-1 envelope proteins are produced as a polyprotein (gp160) which is cleaved into its two subunits gp120 and gp41. Examples of such HIV envelopes is exemplified by but not restricted to SEQ ID NO: 229-366. Mutations can be introduced into the HIV envelope polyprotein (gp160) rendering it resistant to cleavage into its two subunits (gp120 and gp41). Such HIV envelope mutants are considered bona fide envelope antigens suited for introduction of further mutations rendering them non-immunosuppressive.

HIV: As used herein, the term “HIV” refers to all forms, subtypes and variations of the HIV virus, and is synonymous with the older terms for HIV, such as HTLV III and LAV. Various cell lines capable of propagating HIV or permanently infected with the HIV virus have been developed and deposited with the ATCC, including HuT 78 cells and the HuT 78 derivative H9, as well as those having accession numbers CCl 214, TIB 161, CRL 1552 and CRL 8543, which are described in U.S. Pat. No. 4,725,669 and Gallo, Scientific 3 0 American 256:46 (1987).

Vaccination modality: A vaccination modality is defined as any step or usage of the immunogen described in this application for any vaccination purpose or part thereof.

Partial knock out of the immunosuppressive domain: Is defined as at least a 30% reduction of the immunosuppression elicited in the CTLL-2 assay by an dimerized mutant peptide as defined in SEQid 1 as compared to a wildtype dimerized peptide as defined in Segid 369.

According to an embodiment, the inventions concerns a method for making an HIV-1 envelope glycoprotein, which may be expressed on the surface of cells, virus or virus like particles, and which provides reduced immune suppression, allowing said HIV-1 envelope glycoprotein to generate an enhanced immune response as compared to wildtype; said method comprising the steps of:
Selecting at least one HIV envelope glycoprotein among the group consisting of GP41 and GP160;
Modifying the immunosuppressive domain of said envelope glycoprotein, said immunosuppressive domain being localizable by the sequence:

L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉

Where X is any amino acid residue;
Said modifying step comprising mutating or genetically modifying at least one of the amino acids selected among the amino acids X₂-X₁₇, whereby said sequence allows maintaining the ability of expression of said HIV-1 envelope glycoprotein on the surface of cells or virus, and further provides reduced immune suppression of said HIV-1 envelope glycoprotein, and wherein said amino acids after said modifying step are selected among:

X2: Q, R, N, A, G
X3: A, T, V, G, Q, R
X4: R, K, G, A, Q
X5: V, I, L, M, A, G, Q, R
X6: L, A, C, D, E, F, G, H, I, K, M, N, P, Q, R, S, T, V, W, Y
X7: A, T, S, G, Q, R
X8: V, I, L, Q, M, A, G, Q, R
X9: E, K, A, G, Q, R
X10: R, S, T, K, G, N, E, D, A, G, Q
X11: L, A, C, D, E, F, G, H, I, K, M, N, P, Q, R, S, T, V, W, Y
X12: L, V, I, M, R, A, G, Q
X13: K, Q, R, A, E, G, S, H, W, T, V
X14: D, E, N, H, G, A, G, Q, R
X15: Q, R, A, G
X16: Q, A, S, K, T, R, H, G
X17: L, M, S, Q, R, H, I, V, F, A, G

According to an embodiment, the inventions concerns a method for making an HIV-1 envelope glycoprotein, which may be expressed on the surface of cells or virus, and which provides reduced immune suppression, allowing said HIV-1 envelope glycoprotein to generate an enhanced immune response as compared to wildtype; said method comprising the steps of:
Selecting at least one HIV envelope glycoprotein among the group consisting of GP41 and GP160;
Modifying the immunosuppressive domain of said envelope glycoprotein, said immunosuppressive domain being localizable by the sequence:

L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉

Said modifying step comprising mutating or genetically modifying at least one of the amino acids selected among the amino acids X₂-X₁₇ into any amino acid, whereby said sequence allow maintaining the ability to expression on the surface of cells or virus, and further provides reduced immune suppression of said HIV-1 envelope glycoprotein.

According to an embodiment, the inventions concerns a method according to the invention, for making an HIV-1 envelope glycoprotein for medical use, such as therapeutic or prophylactic purpose, preferably for use as a vaccine.

According to an embodiment, the invention concerns a method, for making an HIV-1 envelope glycoprotein for vaccination purposes or for the generation of neutralizing antibodies.

According to an embodiment, the invention concerns a method, wherein said modifying step does not provide a sequence selected among a wild type sequence having immune suppressing properties.

According to an embodiment, the invention concerns a method, wherein said modifying step does not provide a sequence selected among the sequences:

X2: Q, R
X3: A, T
X4: R
X5: V, I, L
X6: L
X7: A
X8: V, I, L, M
X9: E
X10: R, S, T, K, G
X11: Y, L, F
X12: L, I, V
X13: K, Q, R, G, S
X14: D, N
X15: Q
X16: Q, K, R
X17: L, I, F

According to an embodiment, the invention concerns a method, wherein said modifying step does not provide a sequence selected among the sequences:

X2: Q, R
X3: A, T
X4: R
X5: V, I, L
X6: L
X7: A
X8: V, I, L, M
X9: E
X10: R, S, T, K
X11: Y, L, F
X12: L, I
X13: K, Q, R, G
X14: D, N
X15: Q
X16: Q, K, R
X17: L, F

According to an embodiment, the invention concerns a method, wherein the HIV-1 envelope glycoprotein may be expressed on the surface of virus, as said sequence provides the ability of expression on the virus to said HIV-1 envelope glycoprotein.

According to an embodiment, the invention concerns a method, wherein the HIV-1 envelope glycoprotein exhibits fusiogenic activity, as said sequence further provides fusiogenic activity to said HIV-1 envelope glycoprotein.

The method according to any of the previous claims, wherein said modifying step provides a sequence selected among:

X2: Q, R, N, A, G
X3: A, T, V, G, Q, R
X4: R, K, G, A, Q
X5: V, I, L, M, A, G, Q, R
X6: L, A, C, D, E, F, G, H, I, K, M, N, P, Q, R, S, T, V, W, Y
X7: A, T, S, G, Q, R
X8: V, I, L, Q, M, A, G, Q, R
X9: E, K, A, G, Q, R
X10: R, S, T, K, G, N, E, D, A, G, Q
X11: L, A, C, D, E, F, G, H, I, K, M, N, P, Q, R, S, T, V, W, Y
X12: L, V, I, M, R, A, G, Q
X13: K, Q, R, A, E, G, S, H, W, T, V
X14: D, E, N, H, G, A, G, Q, R
X15: Q, R, A, G
X16: Q, A, S, K, T, R, H, G
X17: L, M, S, Q, R, H, I, V, F, A, G

The method according to claim 10, wherein said modifying step provides a sequence selected among:

X2: Q, R, A, G,
X3: A, T, G, Q, R
X4: R, K, G, A, G, Q
X5: V, I, L, A, G, Q, R
X6: L, A, C, D, E, F, G, H, I, K, M, N, P, Q, R, S, T, V, W, Y
X7: A, S, T, G, Q, R
X8: V, I, L, M, A, G, Q, R
X9: E, K, A, G, Q, R
X10: R, S, T, K, G, A, Q
X11: L, A, C, D, E, F, G, H, I, K, M, N, P, Q, R, S, T, V, W, Y
X12: L, V, I, M, A, G, Q, R
X13: K, Q, R, A, E, G, S, H, W, T, V, G
X14: D, N, G, A, Q, R
X15: Q, R, A, G
X16: Q, K, T, R, H, A, G
X17: L, R, I, F, G, Q, A

The method according to claim 11, wherein said modifying step provides a sequence selected among:

X2: Q, R, A, G
X3: A, T, G, Q, R
X4: R, K, A, G, Q
X5: V, I, L, A, G, Q, R
X6: L, A, C, D, E, F, G, H, I, K, M, N, P, Q, R, S, T, V, W, Y
X7: A, S, T, A, G, Q, R
X8: V, I, L, A, G, Q, R
X9: E, K, A, G, Q, R
X10: R, S, T, K, G, A, Q
X11: L, A, C, D, E, F, G, H, I, K, M, N, P, Q, R, S, T, V, W, Y
X12: L, V, I, M, A, G, Q, R
X13: K, Q, R, A, E, G, S, H, W, T, V, A, G
X14: D,N, A, G, Q, R
X15: Q, R, A, G
X16: Q, K, T, R, A, G
X17: L, R, I, F, A, G, Q

According to an embodiment, the invention concerns a method, wherein the first 17 amino acids of said sequence is modified into a sequence selected among:

LQARX₅X₆AVERX₁₁LKDQQL,

wherein:
X₆ and X₁₁ independently are selected among any amino acids, and X₅ is selected among:

X5: V, I, L, M, A, G, Q, R

According to an embodiment, the invention concerns a method, wherein the first 17 amino acids of said sequence is modified into a sequence selected among:

LQAR X₅X₆AVERX₁₁LKDQQL

and further harbors 1 or 2 or 3 point mutations selected among any amino acids.

According to an embodiment, the invention concerns a method, wherein the first 17 amino acids of said sequence is modified into a sequence selected among:

LQAR X₅X₆AVERX₁₁LKDQQL

and further harbors 1 or 2 point mutations selected among:

X2: R, N, A, G
X3: T, V, G, Q, R
X4: K, G, A, Q
X5: V, L, M, A, G, Q, R
X7: T, S, G, Q, R
X8: I, L, Q, M, A, G, Q, R
X9: K, A, G, Q, R
X10: S, T, K, G, N, E, D, A, A, G, Q
X12: V, I, M, R, A, G, Q
X13: Q, R, A, E, G, S, H, W, T, V
X14: E, N, H, G, A, G, Q, R
X15: R, A, G
X16: A, S, K, T, R, H, G
X17: M, S, Q, R, H, I, V, F, A

According to an embodiment, the invention concerns a method, wherein the first 17 amino acids of said sequence is modified into a sequence selected among:

LQARX₅X₆AVERX₁₁LKDQQL,

more preferred among

LQARIX₆AVERX₁₁LKDQQL and
LQARVX₆AVERX₁₁LKDQQL.

According to an embodiment, the invention concerns a method, wherein the first 17 amino acids of said sequence is modified into a sequence selected among:

LQARILAVERGLKDQQL and
LQARIQAVERYLKDQQL.

According to an embodiment, the invention concerns a method according to any of the claims 10 to 17, wherein the first 17 amino acids furthermore harbors 1 or 2 or 3 point mutation(s) in said modified sequence.

According to an embodiment, the invention concerns a method, wherein X₆ is Q.

According to an embodiment, the invention concerns a method, wherein X₁₁ is G.

According to an embodiment, the invention concerns a method, wherein the reduced immune suppression is as measured by a technique selected among the group consisting of CTLL2 and PBMC proliferation inhibition assays.

According to an embodiment, the invention concerns a method, wherein the immune suppression after the modifying step is at least 25% reduced, more preferred at least 40% reduced, compared to before the modifying step.

According to an embodiment, the invention concerns a method, wherein the fusiogenic activity, as measured by a technique for measuring cell-cell fusion, preferably is selected among the group consisting of counting syncytia by light microscopy, resonance energy transfer based assays, and indirect reporter gene using techniques or by measuring infectious titers; alternatively, or in addition, the presence of fusiogenic activity may be indicated by the presence of at least one cell expressing the modified envelope and one cell expressing the receptor and/or coreceptors of HIV being fused together.

According to an embodiment, the invention concerns an HIV-1 envelope glycoprotein obtainable according to the invention.

According to an embodiment, the invention concerns an HIV-1 envelope polypeptide, preferably obtained from an HIV-1 envelope glycoprotein according to the preceding claim, consisting of a sequence:

L1X₂X₃X₄X₅X₆X₇X₈X₉X₁₀X₁₁X₁₂X₁₃X₁₄X₁₅X₁₆X₁₇
wherein X is:
X2: Q, R, N, A, G
X3: A, T, V, G, Q, R
X4: R, K, G, A, Q
X5: V, I, L, M, A, G, Q, R
X6: L, A, C, D, E, F, G, H, I, K, M, N, P, Q, R, S, T, V, W, Y
X7: A, T, S, G, Q, R
X8: V, I, L, Q, M, A, G, Q, R
X9: E, K, A, G, Q, R
X10: R, S, T, K, G, N, E, D, A, A, G, Q
X11: L, A, C, D, E, F, G, H, I, K, M, N, P, Q, R, S, T, V, W, Y
X12: L, V, I, M, R, A, G, Q
X13: K, Q, R, A, E, G, S, H, W, T, V
X14: D, E, N, H, G, A, G, Q, R
X15: Q, R, A, G
X16: Q, A, S, K, T, R, H, G
X17: L, M, S, Q, R, H, I, V, F, A

Subject to the proviso that said sequence allows an HIV-1 envelope glycoprotein to generate an enhanced immune response upon inclusion of said sequence in the immunosuppressive domain of said HIV-1 envelope glycoprotein.

According to an embodiment, the invention concerns an HIV-1 envelope polypeptide consisting of the modified domain of the HIV-1 envelope glycoprotein according to the invention.

The expression “the modified domain” will be used to refer to the formerly immunosuppressive domain which provides reduced immune suppression after the modifying step.

According to an embodiment, the invention concerns an antigen obtainable by selecting a part of an HIV-1 envelope glycoprotein, which HIV-1 envelope glycoprotein is obtainable according to the invention, said part comprising the modified domain of said envelope glycoprotein.

According to an embodiment, the invention concerns an antigen comprising the modified domain of an HIV-1 envelope glycoprotein, which HIV-1 envelope glycoprotein is obtainable according to the invention.

According to an embodiment, the invention concerns an antigen comprising a sequence:

L1X₂X₃X₄X₅X₆X₇X₈X₉X₁₀X₁₁X₁₂X₁₃X₁₄X₁₅X₁₆X₁₇
wherein X is:
X2: Q, R, N, A, G
X3: A, T, V, G, Q, R
X4: R, K, G, A, Q
X5: V, I, L, M, A, G, Q, R
X6: L, A, C, D, E, F, G, H, I, K, M, N, P, Q, R, S, T, V, W, Y
X7: A, T, S, G, Q, R
X8: V, I, L, Q, M, A, G, Q, R
X9: E, K, A, G, Q, R
X10: R, S, T, K, G, N, E, D, A, A, G, Q
X11: L, A, C, D, E, F, G, H, I, K, M, N, P, Q, R, S, T, V, W, Y
X12: L, V, I, M, R, A, G, Q
X13: K, Q, R, A, E, G, S, H, W, T, V
X14: D, E, N, H, G, A, G, Q, R
X15: Q, R, A, G
X16: Q, A, S, K, T, R, H, G
X17: L, M, S, Q, R, H, I, V, F, A

Subject to the proviso that said sequence provides the ability of said antigen to be expressed on the surface of cells or virus, and provides reduced immune suppression to said antigen.

According to an embodiment, the invention concerns an antigen, obtainable using a modifying step comprising modifying with a sequence selected among the sequences Seqid 1 to 228.

According to an embodiment, the invention concerns an antigen furthermore harboring 1 or 2 or 3 or 4 or 5 or 6 or 7 or 8 or 9 or 10 or 11 point mutation(s) in any of the sequences 1 to 228.

According to an embodiment, the invention concerns an antigen, which mediates binding of virus to cells expressing CD4.

According to an embodiment, the invention concerns an antigen, which mediates fusion of virus to host cells.

According to an embodiment, the invention concerns an antigen, which invokes an immune response which causes generation of neutralizing antibodies against HIV.

According to an embodiment, the invention concerns an antigen, wherein said immune response comprises a T-cell response against HIV.

According to an embodiment, the invention concerns an antigen, comprising a sequence of an HIV glycoprotein selected among Seqids 229 to 366, or which is at least 70, preferably at least 80, more preferred at least 90, preferably at least 95 percent, more preferred 99 percent identical to a sequence selected among Seqids 229-366.

According to an embodiment, the invention concerns an antigen according to the invention, which is recombinant or obtained by recombinant technology.

According to an embodiment, the invention concerns a nucleic acid sequence, preferably recombinant, encoding an HIV-1 envelope glycoprotein, an HIV-1 envelope polypeptide or an antigen according to the invention.

According to an embodiment, the invention concerns an isolated eukaryotic expression vector comprising a nucleic acid sequence according to the invention.

According to an embodiment, the invention concerns a vector according to the invention, which is a virus vector, preferably a virus selected among the group consisting of vaccinia virus, measles virus, retroviridae, lentivirus and adeno virus.

According to an embodiment, the invention concerns a vaccine composition for HIV comprising an HIV-1 envelope glycoprotein according to the invention.

According to an embodiment, the invention concerns a vaccine composition for HIV according to the invention comprising a virus like particle (VLP).

According to an embodiment, the invention concerns a vaccine composition for HIV according to the invention wherein the virus like particle is obtained using expression vectors for retroviral gag proteins, and optionally pol proteins, combined with an HIV envelope glycoprotein according to the invention.

According to an embodiment, the invention concerns a vaccine composition for HIV according to the invention wherein the virus like particle is obtained using expression vectors for gamma retrovial gag proteins, and optionally pol proteins, combined with an HIV envelope glycoprotein according to the invention.

According to an embodiment, the invention concerns a vaccine composition for HIV according to the invention wherein the virus like particle is obtained using expression vectors for lentiviral gag proteins, and optionally pol proteins, combined with an HIV envelope glycoprotein according to the invention.

According to an embodiment, the invention concerns a vaccine composition for HIV according to the invention wherein the virus like particle is obtained by the usage of expression vectors for HIV derived gag and pol proteins combined with an HIV envelope glycoprotein according to the invention.

According to an embodiment, the invention concerns a vaccine composition for HIV according to the invention, wherein at least one of the group consisting of gag, gag-pol or said HIV envelope glycoprotein, alone or in combination, has been codon optimized.

According to an embodiment, the invention concerns a vaccine composition for HIV according to the invention, wherein the virus like particle is produced ex vivo in a cell culture.

According to an embodiment, the invention concerns a vaccine composition for HIV according to the invention, wherein the virus like particle is partly or completely assembled ex vivo.

According to an embodiment, the invention concerns a vaccine composition for HIV according to the invention, wherein the virus like particle is generated in vivo in the patient by infection, transfection and/or electroporation by expression vectors.

According to an embodiment, the invention concerns a vaccine composition for HIV according to the invention, comprising a vector derived from a measles virus.

According to an embodiment, the invention concerns a vaccine composition for HIV according to the invention, comprising a vector derived from vaccinia virus.

According to an embodiment, the invention concerns a vaccine composition for HIV according to the invention, comprising an MVA vector.

According to an embodiment, the invention concerns a vaccine composition for HIV according to the invention, comprising an MVA-BN vector.

According to an embodiment, the invention concerns a vaccine composition for HIV according to the invention, comprising a purified glycoprotein.

According to an embodiment, the invention concerns a vaccine composition for HIV according to the invention, comprising an expression vector for DNA vaccination.

According to an embodiment, the invention concerns a method for producing an antibody, said method comprising the steps of:
Administering an entity selected among an HIV-1 envelope glycoprotein, an HIV-1 envelope polypeptide, an antigen, a nucleic acid sequence or a vector according to the invention to a host, such as an animal; and
Obtaining the antibody from said host.

According to an embodiment, the invention concerns an antibody obtainable according to the invention.

According to an embodiment, the invention concerns an antibody, which is specific for an entity selected among an HIV-1 envelope glycoprotein, an HIV-1 envelope polypeptide, an antigen, a nucleic acid sequence or a vector according to the invention.

According to an embodiment, the invention concerns a neutralizing antibodies obtained or identified by the use of at least one HIV-1 envelope glycoprotein according to the invention.

According to an embodiment, the invention concerns a method for manufacturing neutralizing antibodies comprising the use of at least one HIV-1 glycoprotein according to the invention.

According to an embodiment, the invention concerns a method for manufacturing humanized neutralizing antibodies, comprising the use of at least one sequence selected among the sequences 1 to 336.

According to an embodiment, the invention concerns a vaccine composition comprising an entity selected among the group consisting of an HIV-1 envelope glycoprotein, an HIV-1 envelope polypeptide, an antigen, a nucleic acid sequence, a vector and an antibody according to the invention, and in addition at least one excipient, carrier or diluent.

According to an embodiment, the invention concerns a vaccine composition according to the invention, further comprising an at least one adjuvant.

According to an embodiment, the invention concerns a vaccine composition according to the invention, wherein a Glade of HIV have been employed in the production of a number of HIV-1 envelope glycoproteins, HIV-1 envelope polypeptides, antigens, nucleic acid sequences, vectors or antibodies according to the invention, which is comprised in said vaccine, said Glade being selected among the clades A, B, C, D, E, and O, preferably B.

According to an embodiment, the invention concerns a vaccine composition according to the invention, wherein a plurality of clades of HIV have been employed in the production of a number of HIV-1 envelope glycoproteins, HIV-1 envelope polypeptides, antigens, nucleic acid sequences, vectors or antibodies according to the invention, which is comprised in said vaccine.

According to an embodiment, the invention concerns a vaccine composition according to the invention, wherein a plurality of clades of HIV, said clades being selected among A, B, C, D, E and O, have been employed in the production of a number of HIV-1 envelope glycoproteins, HIV-1 envelope polypeptides, antigens, nucleic acid sequences, vectors or antibodies according to the invention, which is comprised in said vaccine.

According to an embodiment, the invention concerns a vaccine composition according to the invention, wherein at least one of each of the HIV clades A, C and D, have been employed in the production of a number of HIV-1 envelope glycoproteins, HIV-1 envelope polypeptides, antigens, nucleic acid sequences, vectors or antibodies according to the invention, which is comprised in said vaccine.

According to an embodiment, the invention concerns a vaccine composition according to the invention, wherein at least one of each of the HIV clades C and E, have been employed in the production of a number of HIV-1 envelope glycoproteins, HIV-1 envelope polypeptides, antigens, nucleic acid sequences, vectors or antibodies according to the invention, which is comprised in said vaccine.

According to an embodiment, the invention concerns a vaccine composition according to the invention, wherein at least one of each of the HIV clades A, B, C, D, E and O, have been employed in the production of a number of HIV-1 envelope glycoproteins, HIV-1 envelope polypeptides, antigens, nucleic acid sequences, vectors or antibodies according to the invention, which is comprised in said vaccine.

According to an embodiment, the invention concerns a method for producing a vaccine composition according to the invention comprising combining at least one HIV-1 envelope glycoprotein, an HIV-1 envelope polypeptide, an antigen, a nucleic acid sequence, a vector or an antibody according to the invention, preferably in purified form, with at least one adjuvant.

According to an embodiment, the invention concerns a pharmaceutical composition comprising an HIV-1 envelope glycoprotein, an HIV-1 envelope polypeptide, an antigen, a nucleic acid sequence, a vector, an antibody or a vaccine composition according to the invention, and at least one pharmaceutically acceptable excipient.

According to an embodiment, the invention concerns a use of an HIV-1 envelope glycoprotein or an HIV-1 envelope polypeptide, or a dimerized version or a trimerized version or a multimerized version of the purified envelope polypeptide according to the invention as an antigen.

According to an embodiment, the invention concerns a use of an HIV-1 envelope glycoprotein, an HIV-1 envelope polypeptide, an antigen, a nucleic acid sequence, a vector or an antibody according to the invention, for a medical purpose, such as for the treatment, amelioration or prevention of a clinical condition, such as for the manufacture of a medicament for the treatment, amelioration or prevention of a clinical condition, such as HIV.

According to an embodiment, the invention concerns a method of treating or ameliorating the symptoms of an individual infected with HIV, or prophylactic treating an individual against HIV infection, comprising administering an amount of HIV-1 envelope glycoprotein, HIV-1 envelope polypeptide, antigen, nucleic acid sequence, vector or vaccine composition according to the invention.

A main aspect and embodiment of the present invention is to provide HIV-1 envelope polypeptide, or part thereof, to be used either as an antigen for HIV vaccination or as an antigen for generation of neutralizing antibodies against HIV. The invention also encompasses biological entities comprising such polypeptides or nucleic acids encoding those, especially viral particles or viral like particles, in particular retroviral, retroviral like, lentiviral or lentiviral like particles. Moreover the invention relates to vaccine compositions, which comprise a polypeptide of the present invention and an adjuvant as well as production methods and kits comprising said vaccine compositions.

Due to the high error rate during replication of the HIV-1 genome, the genetic code for HIV-1 envelope is highly variable, both inside anyone person and even more so across populations. The present invention relates to any variant of the HIV-1 envelope polypeptide, in particular any variant in which gp41, comprise an amino acid sequence encompassing a region of 29 amino acids further comprising the mutated immunosuppressive domain rendering said immunosuppressive domain non-immunosuppressive where furthermore the amino acids at position 1, 22, 23 and 29 are invariable with the amino acid at position 1 always being an L, the amino acid at position 22 an G, the amino acids at position 23 an C and the amino acid at position 29 a C. As such the HIV envelope antigen for making an HIV vaccine or generating neutralizing antibodies against HIV should encompass the following sequence:

Segid 1: L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉

In a further preferred embodiment the antigen should encompass a sequence where the amino acids has been even more restricted to

Segid 2: L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉ where X is
X1: L
X2: Q, R, N, A, G
X3: A, T, V, G, Q, R
X4: R, K, G, A, Q
X5: V, I, L, M, A, G, Q, R
X6: L, A, C, D, E, F, G, H, I, K, M, N, P, Q, R, S, T, V, W, Y
X7: A, T, S, G, Q, R
X8: V, I, L, Q, M, A, G, Q, R
X9: E, K, A, G, Q, R
X10: R, S, T, K, G, N, E, D, A, A, G, Q
X11: L, A, C, D, E, F, G, H, I, K, M, N, P, Q, R, S, T, V, W, Y
X12: L, V, I, M, R, A, G, Q
X13: K, Q, R, A, E, G, S, H, W, T, V
X14: D, E, N, H, G, A, G, Q, R
X15: Q, R, A, G
X16: Q, A, S, K, T, R, H, G
X17: L, M, S, Q, R, H, I, V, F, A
X18: L, M
X19: G, N, S, A, Q, R
X20: I, L, S, A, T, E, V, M, F
X21: W, F
X22: G
X23: C
X24: S, K, T, Q, R, A
X25: G, F, D, N, W
X26: K, N, R, F, M, Q, A, S
X27: L, H, P, A, Q, S, T, W, I, V, F, R
X28: I, V, T
X29: C

In yet another preferred embodiment the antigen should encompass a sequence where the aminoacids has been even further restricted to:

Segid 3: L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉ where X is
X1: L
X2: Q, R, A, G, Q
X3: A, T, G, Q, R
X4: R, K, G, A, G, Q
X5: V, I, L, A, G, Q, R
X6: L, A, C, D, E, F, G, H, I, K, M, N, P, Q, R, S, T, V, W, Y
X7: A, S, T, G, Q, R
X8: V, I, L, M, A, G, Q, R
X9: E, K, A, G, Q, R
X10: R, S, T, K, G, A, Q
X11: L, A, C, D, E, F, G, H, I, K, M, N, P, Q, R, S, T, V, W, Y
X12: L, V, I, M, A, G, Q, R
X13: K, Q, R, A, E, G, S, H, W, T, V, G
X14: D, N, G, A, Q, R
X15: Q, R, A, G
X16: Q, K, T, R, H, A, G
X17: L, R, I, F, G, Q
X18: L, M
X19: G, N, S, R
X20: I, L, S, A, T, E, V, M, F
X21: W
X22: G
X23: C
X24: S, K, T, R, A
X25: G
X26: K, N, R
X27: L, H, P, A, Q, S, T, W, I, V, F, R
X28: I, V
X29: C

In a yet more preferred embodiment the antigen should encompass a sequence where the amino acids has even been further restricted to:

Segid 4: L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉ where X is
X1: L
X2: Q, R, A, G
X3: A, T, G, Q, R
X4: R, K, A, G, Q
X5: V, I, L, A, G, Q, R
X6: L, A, C, D, E, F, G, H, I, K, M, N, P, Q, R, S, T, V, W, Y
X7: A, S, T, A, G, Q, R
X8: V, I, L, A, G, Q, R
X9: E, K, A, G, Q, R
X10: R, S, T, K, G, A, Q
X11: L, A, C, D, E, F, G, H, I, K, M, N, P, Q, R, S, T, V, W, Y
X12: L, V, I, M, A, G, Q, R
X13: K, Q, R, A, E, G, S, H, W, T, V, A, G
X14: D, N, A, G, Q, R
X15: Q, R, A, G
X16: Q, K, T, R, A, G
X17: L, R, I, F, A, G, Q
X18: L
X19: G, N, S, R
X20: I, L, S, V, F
X21: W
X22: G
X23: C
X24: S, K, T, A
X25: G
X26: K, R
X27: L, H, P, A, Q, S, T, W, I, V, F, R
X28: I, V
X29: C

In other embodiments of the present invention the HIV-1 envelope polypeptide or a fragment thereof as defined by seqid nr 229 to 366 has a sequence that is for example at least 40%, such as at least 45%, for example at least 50%, such as at least 55%, for example at least 60%, such as at least 65%, for example at least 67%, such as at least 70%, for example at least 72%, such as at least 75%, for example at least 77%, such as at least 80%, for example at least 81%, such as at least 82%, for example at least 83%, such as at least 84%, for example at least 85%, such as at least 86%, for example at least 87%, such as at least 88%, for example at least 89%, such as at least 90%, for example at least 91%, such as at least 92%, for example at least 93%, such as at least 94%, for example at least 95%, such as at least 96%, for example at least 97%, such as at least 98%, for example at least 99% identical to the amino acid sequence of any polypeptide of the present invention.

According to an embodiment, the following sequence is not part of the invention.

<seqid367; PRT;>
L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉
Where
X2: Q, R
X3: A, T
X4: R
X5: V, I, L
X6: L
X7: A
X8: V, I, L, M
X9: E
X10: R, S, T, K, G
X11: Y, L, F
X12: L, I, V
X13: K, Q, R, G, S
X14: D, N
X15: Q
X16: Q, K, R
X17: L, I, F
<seqid368; PRT;>
L₁X₂X₃X₄X₅X₆X₇X₈X₉X₁₀X₁₁X₁₂X₁₃X₁₄X₁₅X₁₆X₁₇X₁₈X₁₉X₂₀X₂₁G₂₂C₂₃X₂₄X₂₅X₂₆X₂₇X₂₈C₂₉
Where
X2: Q, R
X3: A, T
X4: R
X5: V, I, L
X6: L
X7: A
X8: V, I, L, M
X9: E
X10: R, S, T, K
X11: Y, L, F
X12: L, I
X13: K, Q, R, G
X14: D, N
X15: Q
X16: Q, K, R
X17: L, F
<seqid369; PRT;>
LQARVLAVERYLKDQQLLGIWGC

### Antigen

One aspect of the present invention relates to an antigen, which comprises a peptide derived from an HIV-1 envelope polypeptide of the present invention. In particular, the antigen incorporates a part of HIV-1 envelope polypeptide, including any region of gp41 and/or gp120 and/or gp160, such as for example the transmembrane domain (TM-domain) or the surface subunit (SU). Preferably, the antigen comprises at least one peptide with an amino acid sequence of an HIV-1 envelope polypeptide of the present invention, or a functional homolog thereof having at least 70%, such as at least 80%, for example at least 90% identity to said envelope.

### The Vaccine Composition

The vaccine composition of the invention is in one embodiment capable of eliciting a cellular immune response in the individual. For example, the vaccine composition is capable of eliciting the production in a vaccinated individual of effector T-cells having a cytotoxic effect against HIV-1 infected cells in a subject. In another embodiment, the vaccine composition is capable of eliciting the production in a vaccinated individual of regulatory T-cells having a cytotoxic effect against cells expressing HIV-1 envelope polypeptide or part thereof, and/or antigen presenting cells expressing HIV-1 envelope or part thereof. In another embodiment, the vaccine composition of the present invention is capable of mediating an antibody response in an individual and/or a biological entity.

In a particular embodiment the vaccine composition is to be given against infection with HIV, in particular HIV-1. The present invention therefore also pertains to a vaccine composition which is administered to an animal including a human being, in which the vaccine is capable of eliciting an immune response against a disease caused by a lentivirus, in particular HIV-1. Thus, a vaccine composition of the present invention is capable of eliciting a clinical response in a subject, wherein the clinical response is characterized by a reduced susceptibility, resistance, stabilization, remission or curing/recovery of an HIV infection and/or AIDS.

One embodiment combines anyone of the components of the present invention, including an HIV-1 envelope polypeptide, an antigen, a nucleic acid, an eukaryotic expression vector, and/or a biological entity of the present invention with at least one adjuvant or supplement to produce a vaccine composition.

Yet another embodiment combines any component of the present invention, such as an HIV-1 envelope polypeptide, a trimerized version of the HIV envelope, an antigen, a virus like particle, and/or a biological entity of the present invention with at least one adjuvant or supplement to produce a vaccine composition.

### Antibody

It is one aspect of the present invention to provide antibodies or functional equivalents thereof, such as antigen binding fragments or recombinant proteins specifically recognizing and binding an HIV-1 envelope polypeptide, such as an HIV envelope polypeptide encoded by a gene selected from the group consisting of SEQ ID NO: 229-366. In one embodiment, the antibody, antigen binding fragment, recombinant protein or functional homologue thereof is a neutralizing antibody capable of neutralizing HIV.

The antibodies according to the present invention may be a monoclonal antibody derived from a mammal or a synthetic antibody, such as a single chain antibody or hybrids comprising antibody fragments. Furthermore, the antibody may be mixtures of monoclonal antibodies or artificial polyclonal antibodies. In addition functional equivalents of antibodies may be antibody fragments, in particular epitope binding fragments. Furthermore, antibodies or functional equivalent thereof may be small molecule mimetics, mimicking an antibody.

Polyclonal antibodies is a mixture of antibody molecules recognizing a specific given antigen, hence polyclonal antibodies may recognize different epitopes within said antigen. In general polyclonal antibodies are purified from serum of a mammal, which previously has been immunized with the antigen.

The antibodies according to the present invention may also be recombinant antibodies. Recombinant antibodies are antibodies or fragments thereof or functional equivalents thereof produced using recombinant technology. For example recombinant antibodies may be produced using a synthetic library or by phage display.

Human monoclonal antibodies of the invention can be produced by a variety of techniques, including conventional monoclonal antibody methodology. Although somatic cell hybridization procedures are preferred, in principle, other techniques for producing monoclonal antibody can be employed, e.g., viral or oncogenic transformation of B-lymphocytes or phage display techniques using libraries of human antibody genes.

### Antibody Targets

In one embodiment of the present invention, the antibody, antigen binding fragment or recombinant protein thereof is capable of specifically recognizing and binding an HIV-1 envelope polypeptide, such as an HIV-1 envelope polypeptide selected from anyone of SEQ ID NO: 229-366

Importantly, the present invention encompasses use of an antibody as defined herein, for the manufacture of a medicament for the treatment of a clinical condition as defined herein, such as HIV-1 and/or AIDS. Also, the present invention encompasses methods of treatment of a clinical condition as defined herein, such as HIV-1 and/or AIDS comprising administration of an antibody as described herein to a person in need thereof. The invention also relates to an antibody as defined herein for treatment of said clinical condition. Thus, one aspect of the present invention relates to an antibody, antigen binding fragment or recombinant protein thereof, which is specific for an HIV-1 envelope polypeptide or part thereof as defined herein, and/or a nucleic acid as defined herein, and/or an antigen as defined herein, and/or a biological entity as defined herein. In one embodiment, the antibody, antigen binding fragment or recombinant protein thereof is capable of initiating an immune response against HIV-1 retroviral particles.

All cited references are incorporated by reference.

The accompanying Figure and Examples are provided to explain rather than limit the present invention. It will be clear to the person skilled in the art that aspects, embodiments and claims of the present invention may be combined.

### EXAMPLES

### Example 1

### Cells and Cell Based Assays for Immunosuppression

### Virus Preparation

Supernatant from virus producing cultures will either be purified by sucrose gradient centrifugation. The particles purified by sucrose gradient centrifugation will either be used directly, inactivated by UV treatment or disrupted by 0.6M KCL and 0.5% triton X100 and clarified by centrifugation at 60.000 to 100.000 g for 1 hour.

Assay to measure the immunosuppressive activity of peptides derived from HIV glycoproteins or the glycoproteins from HIV with mutations according to any of the claims:

Abrogation of Immunosuppression of dimerized peptides are considered verified if the dimerized peptides scores positive in either the PBMC or the CTLL-2 assay listed below.

### PBMC Assay

Human Peripheral Blood Mononuclear Cells (PBMC) are prepared freshly from healthy donors. These are stimulated by Con A (5 ug/mL) concomitant to peptide addition at different concentrations (i.e. 25 uM, 50 uM and 100 uM). Cultures are maintained and lymphocyte proliferation is measured 72 hrs later by EdU incorporation and Click-iT labelling with Oregon Green (Invitrogen, Denmark) as recommended by the manufacturer. The degree of activated lymphocytes is proportional to the fluorescence detection.

### CTLL-2 Assay

100.000 CTLL-2 cells are seeded pr. well in a 48 well-plate (Nunc) in 200 uL of medium (RPMI+2 mM L-glutamine+1 mM Na-pyruvat+10% FCS+0.5 ng/mL IL-2) 2 hours later the peptides are added to the wells. 24 h later the cells are labeled using the Click-it reaction kit (Invitrogen cat. #C35002). The fluorescence of the cells is measured on a flow cytometer. The degree of proliferation in each sample is proportional to the detected fluorescence.

Percent inhibition will be calculated as:%Inh.=[control(stim.)−control(unstim.)][exper.(stim.)−exper.(unstim.)]/[control(stim.)−control(unstim.)]×100.

### Example 2

### Peptide Preparation

The peptides can be prepared by different means including, but not limited to solid phase synthesis commonly used for such purposes. The peptides can be dimerized using a cysteine residue either at the N- or C-terminal or in the middle of the peptide or by using any other molecule or atom that is covalently bound to peptide molecules.

The peptides can be coupled to a carrier protein such as BSA by covalent bounds including but not limited to disulfide bridges between the peptide cysteine residues and the carrier protein or through amino groups including those in the side chain or Lysine residues.

For peptides where solubility is an issue Aminoacids will be applayed C-terminally to increase solubility. In particular and GGEKEKEK tail has been used to increase the solubility

### Example 3

### Immunogenicity of Viral Particles

The development of a broad and neutralizing antibody response is vital for a protective HIV-1 vaccine. Protection of macaques against vaginal transmission of a pathogenic HIV-1/SIV chimeric virus by passive infusion of neutralizing antibodies). In addition, the induction of specific and effective cytotoxic T lymphocytes has been shown to be required for infection control. Control of Viremia in Simian Immunodeficiency Virus Infection by CD8+ Lymphocytes).

Thus, the development of vaccine strategies that encompass both arms of the immune system are thus important. Differential MHC I and II antigen presentation is a key factor for initiation of a potent immune response. There appear to be short-comings in antigen presentation when antigens are administered solely as peptides or DNA. In contrast cross-talk between antigen presenting cells and T helper lymphocytes are promoted in vaccine strategies based on either infectious or non-infectious viral particles. A potent induction of cell mediated immunity can be achieved even with whole-killed viral particles. Membrane embedded HIV-1 envelope on the surface of a virus-like particle elicits broader immune responses than soluble envelopes). This is likely a consequence of improved antigen uptake and systemic immunestimulation in macrophages and dendritic cells. Baculovirus-derived human immunodeficiency virus type i virus-like particle activate dendritic cells and induce ex vivo t-cell responses). To achieve potent immunogens viral particles, lentiviral particles, retroviral particles, γ-retroviral particles or any other type of viral or virus like particle may be produced with functional HIV-1 envelope trimers on the surface. These particles can either be produced in vitro (e.g. but not restricted to cell culture) or in vivo (e.g. but not restricted cells in the patient infected by a vaccinia virus like MVA) where they will function as a superior immunogenic particle/antigen.

### Example 4

### Detection of Cell Surface Expression of ENV by Flow Cytometry

The cells are labeled with anti HIV-ENV antibodies through incubation of the cells with the Ab for 45 min on ice. Followed by washing of the unbound Ab with PBS. The cell-anti envelope Ab complex is subsequently incubated with a fluorescent labeled Ab against the primary ENV-binding Ab for 45 min. on ice followed by a second PBS wash. A flow cytometer will be used to detect fluorescence associated with the cells, which is indicative of ENV expression.

### Example 5

### Detection of Incorporation of ENV into Retroviral Particles by Flow Cytometry

Supernatant containing retroviral particles is incubated with cells expressing the CD4 receptor for 45 min. on ice followed by PBS wash. The cells are subsequently labeled with anti HIV-ENV antibodies through incubation of the cells with the Ab for 45 min on ice followed by washing of the unbound Ab with PBS. The cell-anti envelope Ab complex is subsequently incubated with a fluorescent labeled Ab against the primary ENV-binding Ab for 45 min. on ice followed by a second PBS wash. A flow cytometer will be used to detect fluorescence associated with the cells, which is indicative of ENV expression.

### Example 6

### Detection of Fusogenicity of the ENV by Syncytia Assay

An Env-expressing plasmid (which may also expresses egfp marker which may or may not be targeted to the neceus is transfected into 293T cells. Two days later, the ENV-expressing cells are co-cultivated with D17 cells expressing CD4 (10,000 cells pr. square centimeter) and one or more of the HIV coreceptors (ie. CXCR4, CCR5 etc. 10,000 cells pr. square centimeter). Fusogenicity of the ENV protein can be detected by examination of the level of cell-cell fusion in a microscope, either in visible light or by green fluorescence found in the cells. The level of syncyitum formation is reflected by the number of nuclei in syncytia in an arbitrary field of vision. At least two nuclei in one syncytium indicates that fusion has happened albeit at a low level. Thus any mutant of the HIV envelope protein that causes a syncytium containing at least two nuclei is considered fusiogenic. However, in the setting described above syncytia of many dozens of nuclei can be observed. Wt envelope might cause syncyita of more than 100 nuclei to form. Any number of nuclei in synsytia from two to or above the number of nuclei in syncytia caused by wt HIV envelope protein, suggests fusiogenicity of the mutated HIV envelope proteins.

### Example 7

### Production and Test of HIV Mutant VLPs Produced Using an MVA Vaccine

To evaluate the efficacy of a modified vaccinia virus Ankara (MVA) vaccine in the SIV macaque therapeutic immunization model. MVA will selected as a vaccine vector, since it has previously shown to induce strong antiviral immune responses and protection. As viral antigens SIV Gag-Pol and HIV Env will used. The gag/pol and HIV env will be humanized in order to omit tat and rev. Infection of a cell with an MVA vector expressing these proteins will result in the transient production of VLP5 from the cell.

For vaccination of humans the SIV gag/pol can be exchanged with a humanized version of HIV gag/pol [14]

### Example 8

### Functional Test of Conformational Status of Specific HIV Envelope Mutants by

### Titer:

Virions were produced by transfection of 293T cells with a mixture of plasmids that express the envelope protein, gagpol polyprotein from HIV, Rev protein from HIV and a lentiviral vector that expresses the egfp gene. 24 h later the medium was renewed on the cells. The virions were harvested 30 hours post transfection and used to infect D17CXCR4CD4 cells in serial dilutions. Colonies that express egfp were visualized in fluorescent microscope and counted, on the basis of which the titers were estimated.

ISD#19 has a titer of 10⁵ cfu/mL
ISD#4 has a titer of 10³ cfu/mL
Wt HIV envelop has titer of 10⁵ cfu/mL

### Example 9

### Immunosuppression of Selected Peptides

The immunosuppression effect of the ISDs:

The immunes suppression was determined by the effect of peptides corresponding to ISDs on the proliferation of CTLL-2 cells.

Peptides corresponding to the following sequences containing ISDs from either wt, ISD#4 or ISD#19 were synthesized and dimerized by formation of disulfide bonds between the cystein residues.

ISD#4:
LQARILAVERGLKDQQLLGIWGCGGEKEKEK
ISD#19:
LQARIQAVERYLKDQQLLGIWGCGGEKEKEK
Wt:
LQARILAVERYLKDQQLLGIWGCGGEKEKEK

The peptides were used in the following assay:

100.000 CTLL-2 cells were seeded pr. well in a 48 well-plate (Nunc) in 200 uL of medium (RPMI+2 mM L-glutamine+1 mM Na-pyruvat+10% FCS+0.5 ng/mL IL-2) 2 hours later the peptides were added to the wells. 24 h later the cells were labeled using the Click-it reaction kit (Invitrogen cat. #C35002). The fluorescence of the cells was measured on a flow cytometer. The degree of proliferation in each sample were proportional to the detected fluorescence.

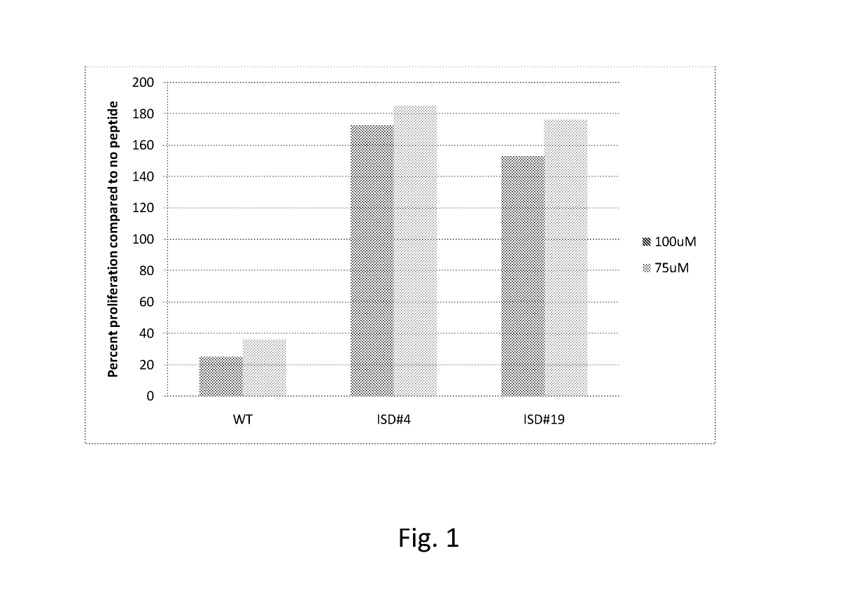

As can be seen in FIG. 1 the wt ISD inhibits the proliferation of the CTLL2 cells at concentrations of 75 or 100 uM. Neither ISD#4 or ISD#19 inhibits the proliferation.

### REFERENCES

1. Sapir, A., et al., Viral and developmental cell fusion mechanisms: conservation and divergence. Dev Cell, 2008. 14(1): p. 11-21.
2. Cianciolo, G. J., et al., Murine malignant cells synthesize a 19,000-dalton protein that is physicochemically and antigenically related to the immunosuppressive retroviral protein, P15E. J Exp Med, 1983. 158(3): p. 885-900.
3. Hebebrand, L. C., et al., Inhibition of human lymphocyte mitogen and antigen response by a 15,000-dalton protein from feline leukemia virus. Cancer Res, 1979. 39(2 Pt 1): p. 443-7.
4. Cianciolo, G. J., et al., Macrophage accumulation in mice is inhibited by low molecular weight products from murine leukemia viruses. J Immunol, 1980. 124(6): p. 2900-5.
5. Mangeney, M. and T. Heidmann, Tumor cells expressing a retroviral envelope escape immune rejection in vivo. Proc Natl Acad Sci USA, 1998. 95(25): p. 14920-5.
6. Mangeney, M., et al., Placental syncytins: Genetic disjunction between the fusogenic and immunosuppressive activity of retroviral envelope proteins. Proc Natl Acad Sci USA, 2007. 104(51): p. 20534-9.
7. Cianciolo, G. J., et al., Inhibition of lymphocyte proliferation by a synthetic peptide homologous to retroviral envelope proteins. Science, 1985. 230(4724): p. 453-5.
8. Cianciolo, G. J., H. Bogerd, and R. Snyderman, Human retrovirus-related synthetic peptides inhibit T lymphocyte proliferation. Immunol Lett, 1988. 19(1): p. 7-13.
9. Yaddanapudi, K., et al., Implication of a retrovirus-like glycoprotein peptide in the immunopathogenesis of Ebola and Marburg viruses. Faseb J, 2006. 20(14): p. 2519-30.
10. Haraguchi, S., et al. Differential modulation of Th1-and Th2-related cytokine mRNA expression by a synthetic peptide homologous to a conserved domain within retroviral envelope protein. Proc Natl Acad Sci USA, 1995. 92, 3611-15.
11. Harrell, R. A., et al Cianciolo. Suppression of the respiratory burst of human monocytes by a synthetic peptide homologous to envelope proteins of human and animal retroviruses. J Immunol, 1986. 136, 3517-520.
12. Kleinerman, E. S., et al. Lachman. A synthetic peptidehomologous to the envelope proteins of retroviruses inhibits monocyte-mediated killing by inactivatinginterleukin 1. J Immunol, 1987. 139, 2329-337.
13. Schlecht-Louf G. et al. Retroviral infection in vivo requires an immune escape virulence factor encrypted in the envelope protein of oncoretroviruses. Proc Natl Acad Sci USA. 2010 Feb. 23; 107(8):3782-7.
14 Uberla K. et al. Therapeutic immunization with Modified Vaccinia Virus Ankara (MVA) vaccines in SIV-infected rhesus monkeys undergoing antiretroviral therapy. J Med. Primatol. 2007 February; 36(1):2-9.

1.-75. (canceled)

76. A method for making an HIV-1 envelope glycoprotein, said method comprising the steps of:
selecting at least one HIV envelope glycoprotein from the group consisting of GP41 and GP160; and
modifying the immunosuppressive domain of said envelope glycoprotein, said immunosuppressive domain being localizable by the sequence:
LX₂X₃X₄X₅X₆X₇X₈X₉X₁₀X₁₁X₁₂X₁₃X₁₄X₁₅X₁₆X₁₇X₁₈X₁₉X₂₀X₂₁GCX₂₄X₂₅X₂₆X₂₇X₂₈C
(SEQ ID NO:1), wherein X₂-X₂₁ and X₂₄-X₂₈ are independently any amino acid residue; said modifying step comprising mutating or genetically modifying at least one amino acid selected from the group consisting of amino acids X₂-X₁₇, wherein said amino acids after said modifying step are selected from the following respective groups:
X₂: Q, R, N, A, G
X₃: A, T, V, G, Q, R
X₄: R, K, G, A, Q
X₅: V, I, L, M, A, G, Q, R
X₆: L, A, C, D, E, F, G, H, I, K, M, N, P, Q, R, S, T, V, W, Y
X₇: A, T, S, G, Q, R
X₈: V, I, L, Q, M, A, G, Q, R
X₉: E, K, A, G, Q, R
X₁₀: R, S, T, K, G, N, E, D, A, G, Q
X₁₁: L, A, C, D, E, F, G, H, I, K, M, N, P, Q, R, S, T, V, W, Y
X₁₂: L, V, I, M, R, A, G, Q
X₁₃: K, Q, R, A, E, G, S, H, W, T, V
X₁₄: D, E, N, H, G, A, G, Q, R
X₁₅: Q, R, A, G
X₁₆: Q, A, S, K, T, R, H, G
X₁₇: L, M, S, Q, R, H, I, V, F, A, G;
wherein said glycoprotein is capable of being expressed on the surface of a cell, virus, or virus-like particle and provides reduced immune suppression, allowing said HIV-1 envelope glycoprotein to generate an enhanced immune response as compared to the corresponding wild-type HIV-1 envelope glycoprotein.

77. The method according to claim 76, further comprising using said HIV-1 envelope glycoprotein for the production of a vaccine.

78. The method according to claim 76, further comprising using said HIV-1 envelope glycoprotein is for the generation of neutralizing antibodies.

79. The method according to claim 76, wherein said modifying step does not provide a wild type sequence having immune suppressing properties.

80. The method according to claim 76, wherein said modifying step does not provide a sequence comprising any of the following amino acids:
X₂: Q, R
X₃: A, T
X₄: R
X₅: V, I, L
X₆: L
X₇: A
X₈: V, I, L, M
X₉: E
X₁₀: R, S, T, K, G
X₁₁: Y, L, F
X₁₂: L, I, V
X₁₃: K, Q, R, G, S
X₁₄: D, N
X₁₅: Q
X₁₆: Q, K, R
X₁₇: L, I, F.

81. The method according to claim 76, wherein the HIV-1 envelope glycoprotein exhibits fusogenic activity provided by said sequence.

82. The method according to claim 76, wherein the first 17 amino acids of said sequence are modified to LQARX₅X₆AVERX₁₁LKDQQL, and wherein X₆ and X₁₁ are independently any amino acids, and X₅ is selected from V, I, L, M, A, G, Q, and R.

83. The method according to claim 76, wherein the first 17 amino acids of said sequence are modified to LQARX₅X₆AVERX₁₁LKDQQL, and further harbor 1 or 2 or 3 point mutations represented by any amino acids.

84. The method according to claim 76, wherein the first 17 amino acids of said sequence are modified to LQARX₅X₆AVERX₁₁LKDQQL, and further harbors 1 or 2 point mutations selected from any of the following amino acids:
X₂: R, N, A, G
X₃: T, V, G, Q, R
X₄: K, G, A, Q
X₅: V, L, M, A, G, Q, R
X₇: T, S, G, Q, R
X₈: I, L, Q, M, A, G, Q, R
X₉: K, A, G, Q, R
X₁₀: S, T, K, G, N, E, D, A, A, G, Q
X₁₂: V, I, M, R, A, G, Q
X₁₃: Q, R, A, E, G, S, H, W, T, V
X₁₄: E, N, H, G, A, G, Q, R
X₁₅: R, A, G
X₁₆: A, S, K, T, R, H, G
X₁₇: M, S, Q, R, H, I, V, F, A.

85. The method according to claim 76, wherein the first 17 amino acids of said sequence are modified to a sequence selected from the group consisting of:
LQARX₅X₆AVERX₁₁LKDQQL,
LQARIX₆AVERX₁₁LKDQQL, and
LQARVX₆AVERX₁₁LKDQQL.

86. The method according to claim 76, wherein the first 17 amino acids of said sequence are modified to LQARILAVERGLKDQQL or LQARIQAVERYLKDQQL.

87. The method according to claim 76, wherein the first 17 amino acids furthermore harbor 1, 2, or 3 point mutations in said modified sequence.

88. The method according to claim 76, wherein the reduced immune suppression is measured by a technique selected from the group consisting of CTLL2 and PBMC proliferation inhibition assays.

89. The method according to claim 76, wherein the immune suppression after the modifying step is at least 25% reduced compared to before the modifying step.

90. An antigen comprising the amino acid sequence:
LX₂X₃X₄X₅X₆X₇X₈X₉X₁₀X₁₁X₁₂X₁₃X₁₄X₁₅X₁₆X₁₇
wherein X₂-X₁₇ are independently selected from the following amino acids:
X₂: Q, R, N, A, G
X₃: A, T, V, G, Q, R
X₄: R, K, G, A, Q
X₅: V, I, L, M, A, G, Q, R
X₆: L, A, C, D, E, F, G, H, I, K, M, N, P, Q, R, S, T, V, W, Y
X₇: A, T, S, G, Q, R
X₈: V, I, L, Q, M, A, G, Q, R
X₉: E, K, A, G, Q, R
X₁₀: R, S, T, K, G, N, E, D, A, A, G, Q
X₁₁: L, A, C, D, E, F, G, H, I, K, M, N, P, Q, R, S, T, V, W, Y
X₁₂: L, V, I, M, R, A, G, Q
X₁₃: K, Q, R, A, E, G, S, H, W, T, V
X₁₄: D, E, N, H, G, A, G, Q, R
X₁₅: Q, R, A, G
X₁₆: Q, A, S, K, T, R, H, G
X₁₇: L, M, S, Q, R, H, I, V, F, A
subject to the proviso that said sequence provides the ability of said antigen to be expressed on the surface of a cell, virus, or virus-like particle, and said antigen is capable of generating an immune response.

91. The antigen according to claim 90, wherein said antigen is obtainable using a method comprising modifying a wild-type antigen to include a sequence selected from the group consisting of SEQ ID NOS:1 to 228.

92. The antigen according to claim 90, wherein said antigen is obtainable using a method comprising modifying a wild-type antigen to include a sequence selected from the group consisting of SEQ ID NOS:1 to 228, wherein said sequence is modified to comprise 1, 2, 3, 4, or 5 point mutations.

93. The antigen according to claim 90, wherein said antigen mediates binding of a virus to a cell expressing CD4.

94. The antigen according to claim 90, which invokes an immune response which causes generation of neutralizing antibodies against HIV.

95. The antigen according to claim 90, comprising a sequence of an HIV glycoprotein selected from the group consisting of SEQ ID NOS:229 to 366, or comprising a sequence which is at least 70 percent identical to a sequence selected from the group consisting of SEQ ID NOS:229 to 366.

96. The antigen according to claim 90, which is recombinant or obtained by recombinant technology.

97. A vaccine composition for HIV comprising an HIV-1 envelope glycoprotein obtainable according to the method of claim 1.

98. The vaccine composition for HIV according to claim 97, further comprising a virus like particle (VLP), wherein the virus like particle is obtained using an expression vector for a retroviral gag protein, and optionally using an expression vector for a retroviral pol protein.
